(19) United States
(12) Patent Application Publication
(10) Pub. No.: US 2021/0121431 A1
(43) Pub. Date: Apr. 29, 2021

(54) NOVEL COMPOSITIONS AND METHODS FOR TREATING CUTANEOUS ULCERS AND NON-HEALING CUTANEOUS WOUNDS USING NITRIC OXIDE-DONATING PROSTAGLANDIN F-2 -ALPHA ANALOGS

(71) Applicant: Warren J. Scherer, Lutz, FL (US)

(72) Inventor: Warren J. Scherer, Lutz, FL (US)

(21) Appl. No.: 16/666,294

(22) Filed: Oct. 28, 2019

(51) Int. Cl. A61K 31/216; A61K 9/00; A61K 9/06; A61P 17/02

(52) U.S. Cl. CPC A61K 31/216; A61P 17/02; A61K 9/06; A61K 9/0014

(57) ABSTRACT

Cutaneous ulcers and non-healing skin wounds are a route for infection and represent a source of significant morbidity and mortality for humans. The present disclosure provides methods and compositions for treating, healing or preventing cutaneous ulcers and non-healing cutaneous wounds via the topical application of an effective amount of a nitric oxide (NO)-donating prostaglandin F2-alpha (PGF2-alpha) analog directly to the affected ulcer or non-healing cutaneous wound of a human in need of such treatment

### FIELD OF THE INVENTION

The present invention relates to methods and compositions for treating, curing, or preventing cutaneous ulcers and non-healing cutaneous wounds. In particular, the present invention relates to topically applying a NO-donating PGF2-alpha analog, admixed with a pharmacologically acceptable carrier analog to treat or prevent cutaneous ulcers and non-healing skin wounds.

### BACKGROUND OF THE INVENTION

In humans, cutaneous ulcers and poorly-healing wounds serve as a route for infection and cause of significant patient morbidity and mortality, often resulting in limb amputation or death from sepsis (Zhang, et al. 2017). In type I diabetes mellitus or type II diabetes mellitus, the vast majority of cutaneous ulcers and non-healing wounds occur as a result of micro-vascular compromise of the lower extremities (Sinwar, 2015). Other causes of cutaneous ulceration and delayed wound healing include autoimmune vasculopathy, peripheral artery disease, sickle cell disease and impaired venous return (venous stasis ulcers). Healing of cutaneous ulcers and non-healing cutaneous wounds occurs through the formation of granulation tissue. Granulation tissue is new connective tissue and microscopic blood vessels that form on the surface of the wound during the healing process. Deposition of connective tissue components such as collagen and extracellular matrix is dependent upon the proliferation and proper functioning of fibroblasts and myofibroblasts. Fibroblast and myofibroblasts depend upon oxygenation from adequate blood flow in order to produce connective tissue.

A method that acts to simultaneously improve both blood flow and fibroblast proliferation would therefore be beneficial to humans in need of such treatment. The present disclosure describes a method of treating, healing or preventing cutaneous ulcers or non-healing cutaneous wounds via the topical application of a nitric oxide-donating prostaglandin F2-alpha (PGF2-alpha) analog directly to the affected ulcer or non-healing cutaneous wound of a human in need of such treatment

Healing of cutaneous ulcers and wounds requires tissue oxygenation ideally provided by adequate blood flow (Alizadeh, 2007). Nitric oxide is an important endogenous, gaseous, mediator that promotes tissue blood flow via potent vasodilation (Palmer, et al. 1987; Ignarro, et al. 1987).

Endogenous NO is produced from L-arginine by the enzyme nitric oxide synthase (NOS) (Palmer, et al. 1988). In vascular tissue, NO is produced by vascular endothelial cells via the action of the enzyme endothelial nitric oxide synthase (eNOS) (Forstermann, et al. 2006).

In addition to its direct vasodilatory effects, NO has also been shown to improve circulation by inhibiting platelet adhesion and aggregation (Salvemini, et al. 1989, Kalinowski, et al. 2002).

The role played by NO in the pathogenesis of cutaneous ulcers and non-healing wounds has been examined by many investigators. It has been shown in a rat model of diabetic skin ulcer, that upregulation of eNOS accelerated vascularization and increased blood supply to produce rapid wound healing (Yang, et al 2016). In peripheral arterial disease, microvascular dysfunction resulting in lower extremity ulceration has been shown to be linked with impaired NO-associated vasodilatory functioning (Hodges, et al. 2015). Because previous studies have shown that NO production is decreased in diabetic patients and because the cells of these patients have reduced sensitivity to NO (Burrow, et al. 2007), Mikaili, et al have hypothesized that applying exogenous NO alone as nitroglycerine (isosorbide dinitrate) could, in theory, provide compensatory amounts of NO to promote healing of skin ulcers (Mikaili, et al. 2014). Given the key role that NO plays in wound healing, it has even been proposed that wound fluid nitrate levels (an indicator of NO activity) could qualify as a biomarker in the management of skin ulcers (Boykin, 2010). Other authors have determined that inadequate cutaneous NO production, due to eNOS genetic mutations is associated with increased diabetic cutaneous vascular complications (Dong J, et al. 2018; Sadati, et al. 2018), supporting the role of NO in wound healing.

Closure and healing of cutaneous ulcers and non-healing skin wounds requires the proliferation of fibroblasts and myofibroblasts. Fibroblasts and myofibroblasts, whose proliferation are highly dependent upon oxygenation, are the main cells that produce granulation tissue and extracellular matrix that contribute to wound healing.

PGF2-alpha and PGF2-alpha analogs have been shown to stimulate the proliferation of fibroblasts and myofibroblasts (Laengle, et al 2006), collagen production (Ding, et al. 2012) and fibrosis (Oga, et al. 2009; Kanno, et al 2013; Bastiaansen-Jenniskens, et al. 2013; Scherer, 2018; Olman M A, 2009) in vitro and in vivo. PGF2-alpha and PGF2-alpha analogs have also been shown to increase contraction of the collagen matrix on which fibroblasts are growing in vitro, thus theoretically promoting wound closure in vivo (Liu Y, et al. 2008; Liu Y, et al 2006; Rayan G M, et al. 1996). Additionally, PGF2-alpha promotes calcium-mediated electrical communication between gap junctionally-coupled fibroblasts (Harks, et al. 2003).

From these findings, one could conclude that a single method of treating cutaneous ulcers and non-healing skin wounds that simultaneously increases cutaneous blood flow and promotes fibroblast/myofibroblast proliferation, wound contraction, collagen production and fibrosis would be beneficial to humans in need of such treatment.

Recently, novel compounds that both release NO and act as PGF2-alpha analogs have been developed for the treatment of glaucoma and ocular hypertension (Borghi, et al. 2010; Impagnatiello, et al. 2011; Krauss, et al. 2011; Impagnatiello, et al. 2015; Garcia, et al. 2016).

Given the role of NO in the pathophysiology of cutaneous ulcers and the requirements for healing (vascularization, oxygenation and fibroblast proliferation), it is proposed in the present disclosure that direct, topical application of an effective amount of a NO-donating PGF2-alpha analog, admixed with a pharmacologically acceptable carrier, would be effective in promoting the healing, treating, curing or preventing cutaneous ulcers and non-healing skin wounds that occur as a result of, but not limited to, the following conditions: type 1 or type 2 diabetes mellitus, peripheral artery disease, venous stasis, sickle cell disease, autoimmune vasculopathy.

There is a need to treat, cure, and prevent cutaneous ulcers and/or non-healing cutaneous wounds.

### BRIEF SUMMARY OF THE INVENTION

The present disclosure provides for a pharmaceutical composition for topical application. The composition may comprise and effective amount of a NO-donating PGF2-alpha analog, admixed with a pharmacologically acceptable carrier.

The pharmaceutical composition may further comprise an effective amount of vascular endothelial growth factor (VEGF).

The pharmaceutical composition may further comprise an effective amount of epidermal growth factor (EGF).

The pharmaceutical composition may further comprise an effective amount of a local anesthetic agent.

The pharmaceutical composition may further comprise an effective amount of a steroid.

The pharmaceutical composition may further comprise an effective amount of a non-steroidal anti-inflammatory agent.

The pharmaceutical composition may further comprise an effective amount of capsaicin.

The pharmaceutical composition may further comprise an effective amount of a prostaglandin transporter inhibitor.

The pharmaceutical composition may further comprise an effective amount of an antibiotic.

The pharmaceutical composition may further comprise an effective amount of an antiviral.

The pharmaceutical composition may further comprise an effective amount of an up-regulator or activator of endogenous nitric oxide synthase.

The pharmaceutical composition may further comprise aloe.

The pharmaceutical composition may further comprise a moisturizer.

The pharmaceutical composition may further comprise a sunscreen.

The present disclosure also provides for a method of treating or preventing cutaneous ulcers and/or non-healing cutaneous wounds. The cutaneous ulcers and/or non-healing cutaneous wounds may be caused by or associated with type I or type II diabetes mellitus, peripheral arterial disease, atherosclerosis, autoimmune vasculopathy, sickle cell-related vasculopathy, venous stasis ulcers or any other cause of compromised cutaneous vascular circulation. The method may comprise topically applying an effective amount of a NO-donating PGF2-alpha analog to an affected area of the skin of the subject.

In certain embodiments of the pharmacological composition, the NO-donating PGF2-alpha analog may be latanoprostene bunod, or salts thereof.

In another embodiment of the pharmacological composition, the NO-donating PGF2-alpha analog may be [(S,E)-1-((1R,2R,3S,5R)-2-((Z)-7-(ethylamino)-7-oxohept-2-enyl)-3,5-dihydroxycyclopentyl)-5-phenylpent-1-en-3-yl 6-(nitrooxy) hexanoate, salts.

In another embodiment of the pharmacological composition, the NO-donating PGF2-alpha analog may include latanoprostene bunod and [(S,E)-1-((1R,2R,3S,5R)-2-((Z)-7-(ethylamino)-7-oxohept-2-enyl)-3,5-dihydroxycyclopentyl)-5-phenylpent-1-en-3-yl 6-(nitrooxy) hexanoate, salts thereof or combinations thereof.

In certain embodiments of the pharmacological composition, non-limiting examples of the prostaglandin moiety of the NO-donating PGF2-alpha analog may include, but are not limited to, prostaglandin-F2-alpha, latanoprost, bimatoprost, travoprost, tafluprost or unoprotone, salts thereof or combinations thereof.

The prostaglandin moiety may be present in an amount ranging from 0.005% to 0.05% by weight (wt %), from 0.01 wt % to 0.025 wt %, from 0.02 wt % to 0.025 wt %, from 0.25 wt % to 0.5 wt %, from 0.5 wt % to 1.0 wt %, relative to the total weight of the composition.

In certain embodiments, in the pharmaceutical composition(s), the NO-donating PGF2-alpha analog is latanoprostene bunod.

### DETAILED DESCRIPTION OF THE INVENTION

The present disclosure provides methods and compositions for treating, healing or preventing cutaneous ulcers and non-healing cutaneous wounds in humans in need of such treatment. In certain embodiments, an effective amount of a nitric oxide (NO)-donating prostaglandin F2-alpha (PGF2-alpha) analog or a pharmaceutically acceptable salt or derivative thereof, is administered directly to the affected ulcer or non-healing cutaneous wound of a subject, e.g., topically.

A NO-donating PGF2-alpha analog is a molecule that both releases a biologically active form of NO and has a prostaglandin F2-alpha moiety that is biologically active at cellular prostaglandin (FP) receptors.

In certain embodiments, the present composition comprises an effective amount of a NO-donating PGF2-alpha analog (e.g. latanoprostene bunod). In one embodiment, the present composition comprises one, two or three structurally-distinct NO-donating PGF2-alpha analogs.

In certain embodiments, a subject is treated with a NO-donating PGF2-alpha analog, or a pharmacologically acceptable salt thereof or solvate thereof, or a physiologically functional derivative thereof, via topical, intravenous, oral, transdermal, or intranasal administration.

Also encompassed by the present disclosure is a method of treating or preventing cutaneous ulcers and non-healing cutaneous wounds. The method may comprise topically applying an effective amount of a NO-donating PGF2-alpha analog to an affected area of the skin of a subject.

The present disclosure provides for a method of treating or preventing cutaneous ulcers and non-healing cutaneous wounds, the method comprising topically applying a pharmaceutical composition comprising an effective amount of a NO-donating PGF2-alpha analog to an affected area of the skin of a subject, wherein the area is affected by a cutaneous ulcers and/or a non-healing cutaneous wound.

In certain embodiments, the present composition comprises an effective amount of a NO-donating PGF2-alpha analog.

In certain embodiments, preventing cutaneous ulcers and/or non-healing cutaneous wounds, is/are treated or prevented by administering a NO-donating PGF2-alpha analog to a subject, via topical, intravenous, oral, transdermal or intranasal administration.

The present active agent may be formulated into a cosmetic, pharmaceutical, and/or dermatological composition for treating cutaneous ulcers and/or non-healing cutaneous wounds.

The present compositions may be applied topically to the face, the neck, the hair, the mucous membranes and the nails, major folds, or any other area of the body skin.

The compositions of the invention may be administered either via a local route, e.g., topically or by subcutaneous and/or intradermal injection, or via a systemic or general route, e.g., orally and/or by intramuscular injection.

The present agent/composition may be administered therapeutically to achieve a therapeutic benefit (“treating”) or prophylactically to achieve a prophylactic benefit (“preventing”). By therapeutic benefit is meant eradication or amelioration of cutaneous ulcers and/or non-healing cutaneous wounds being treated, and/or eradication or amelioration of one or more of the symptoms associated with cutaneous ulcers and/or non-healing cutaneous wounds. By prophylactic benefit is meant prevention or delay of the onset of cutaneous ulcers and/or non-healing cutaneous wounds, including cutaneous ulcers and/or non-healing cutaneous wounds associated with type I or type II diabetes mellitus, peripheral arterial disease, atherosclerosis, autoimmune vasculopathy, sickle cell-related vasculopathy, venous stasis ulcers or any other cause of compromised cutaneous vascular circulation, and/or prevention or delay of the onset of one or more of the symptoms associated with cutaneous ulcers and/or non-healing cutaneous wounds, caused by or associated with type I or type II diabetes mellitus, peripheral arterial disease, atherosclerosis, autoimmune vasculopathy, sickle cell-related vasculopathy, venous stasis ulcers or any other cause of compromised cutaneous vascular circulation. In certain embodiments, an effective amount of the present agent/composition to be administered prevents cutaneous ulcers and/or non-healing cutaneous wounds from developing or being exacerbated into more serious conditions.

“Treating” or “treatment” of a state, disorder or condition includes: (1) preventing or delaying the appearance of clinical symptoms of the state, disorder, or condition developing in a person who may be afflicted with or predisposed to the state, disorder or condition but does not yet experience or display clinical symptoms of the state, disorder or condition; or (2) inhibiting the state, disorder or condition, i.e., arresting, reducing or delaying the development of the disease or a relapse thereof (in case of maintenance treatment) or at least one clinical symptom, sign, or test, thereof; or (3) relieving the disease, i.e., causing regression of the state, disorder or condition or at least one of its clinical or sub-clinical symptoms or signs.

The benefit to a subject to be treated is either statistically significant or at least perceptible to the patient or to the physician.

An effective amount of an agent/drug refers to a therapeutically effective amount or a prophylactically effective amount. A “prophylactically effective amount” refers to an amount effective, at dosages and for periods of time necessary, to achieve the desired prophylactic result. In certain embodiments, since a prophylactic dose is used in subjects prior to or at an earlier stage of a disorder, the prophylactically effective amount is less than the therapeutically effective amount. In certain embodiments, the prophylactically effective amount is similar to, identical to, or more than, the therapeutically effective amount. A therapeutically effective amount of a drug is an amount effective to demonstrate a desired activity of the drug. A therapeutically effective amount may vary depending on the compound, the disorder and its severity and the age, weight, physical condition and responsiveness of the subject to be treated. In certain embodiments, an effective amount of a NO-donating PGF2-alpha analog, or a pharmaceutically acceptable salt or solvate thereof, or a physiologically functional derivative thereof, is an amount effective to prevent or delay the onset of cutaneous ulcers and/or non-healing cutaneous wounds, and/or effective to alleviate, one or more of the symptoms of cutaneous ulcers and/or non-healing cutaneous wounds.

In certain embodiments, the present agent, such as a NO-donating PGF2-alpha analog (e.g., latanoprostene bunod), or a pharmaceutically acceptable salt or solvate thereof, or a derivative thereof, is administered in a composition comprising a pharmaceutically acceptable carrier, excipient or diluent. Also provided herein is a pharmaceutical composition that comprises a NO-donating PGF2-alpha analog (e.g., latanoprostene bunod), or a pharmaceutically acceptable salt or solvate thereof, or a derivative thereof, and a pharmaceutically acceptable carrier, excipient or diluent, for use in the prophylactic and/or therapeutic treatment of cutaneous ulcers and/or non-healing cutaneous wounds. In certain embodiments, the present disclosure relates to compositions for topical application to the human skin.

The compounds used in the present methods include all hydrates, solvates, and complexes of the compounds. In cases in which compounds have unsaturated carbon-carbon double bonds, both the cis (Z) and trans (E) isomers are within the scope of this invention. If a chiral center or another form of an isomeric center is present in a compound of the present invention, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The compounds may be in racemic form or as individual enantiomers. The enantiomers can be separated using known techniques, such as those described in Pure and Applied Chemistry 69, 1469-1474, (1997) IUPAC. In cases wherein compounds may exist in tautomeric forms, such as keto-enol tautomers, each tautomeric form is contemplated as being included within this invention whether existing in equilibrium or predominantly in one form.

The present disclosure is also intended to include use of all isotopes of atoms occurring on the compounds disclosed herein. Isotopes include those atoms having the same atomic number but different mass numbers. Isotopically-labeled compounds can generally be prepared by conventional techniques known to those skilled in the art in place of the non-labeled reagents employed.

The compounds of the instant invention may be in a salt form. As used herein, a “salt” is a salt of the present compound which has been modified by making acid or base, salts of the compounds. The salt may be pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately treating a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The present methods also encompass administering a physiologically functional derivative of the present compound. As used herein, the term “physiologically functional derivative” refers to a compound (e.g, a drug precursor) that is transformed in vivo to yield the present compound or its active metabolite, or a pharmaceutically acceptable salt, hydrate or solvate of the compound. The transformation may occur by various mechanisms (e.g., by metabolic or chemical processes), such as, for example, through hydrolysis. Prodrugs are such derivatives, and a discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

### Nitric Oxide-Donating Prostaglandin-F2-Alpha Analogs

The present composition may comprise one or more nitric oxide-donating prostaglandin-F2-alpha (NO-donating PGF2-alpha) analogs.

In one embodiment, the NO-donating PGF2-alpha analog is lantanoprostene bunod, or salts thereof.

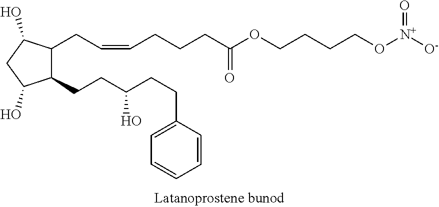 [SMILES: O=C(CCC/C=C\CC1[C@@H](CC[C@@H](O)CCc2ccccc2)[C@H](O)C[C@@H]1O)OCCCCO[N+](=O)[O-]]

In another embodiment, the NO-donating PGF2-alpha analog is [(S,E)-1-((1R,2R,3S,5R)-2-((Z)-7-(ethylamino)-7-oxohept-2-enyl)-3,5-dihydroxycyclopentyl)-5-phenylpent-1-en-3-yl 6-(nitrooxy) hexanoate, or salts thereof.

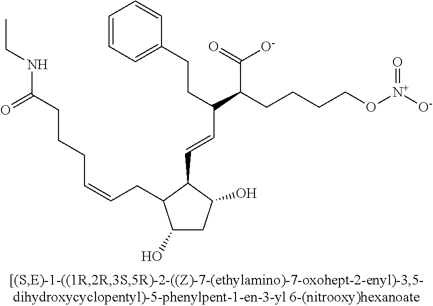 [SMILES: CCNC(=O)CCC/C=C\CC1[C@@H](/C=C/C(CCc2ccccc2)[C@H](CCCCO[N+](=O)[O-])C(=O)[O-])[C@H](O)C[C@@H]1O]

In another embodiment of the pharmacological composition, the NO-donating PGF2-alpha analog may include latanoprostene bunod and [(S,E)-1-((1R,2R,3S,5R)-2-((Z)-7-(ethylamino)-7-oxohept-2-enyl)-3,5-dihydroxycyclopentyl)-5-phenylpent-1-en-3-yl 6-(nitrooxy) hexanoate, salts thereof or combinations thereof.

In certain embodiments, the NO-donating PGF2-alpha analog is in a purified form. In certain embodiments, the purified NO-donating PGF2-alpha analog used in the present compositions and methods comprise (consist of, or consist essentially of) of the trans isomer.

The NO-donating PGF2-alpha analog may be chemically synthesized, or may be of plant, animal, or bacterial origin.

In certain embodiments, a NO-donating PGF2-alpha analog or derivative is administered in a therapeutically equivalent amount. In certain embodiments, a NO-donating PGF2-alpha analog or derivative is administered at a dose higher or lower than a therapeutically equivalent amount of NO-donating PGF2-alpha analog.

In certain embodiments, the present composition comprises about 0.005% to about 20% by weight, about 0.1% to about 18% by weight, about 0.2% to about 15% by weight, about 0.3% to about 10% by weight, about 0.5% to about 8% by weight, about 0.3%, about 0.4%, about 0.45%, about 0.5%, about 0.55% or about 0.6%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5% or about 10.0%, by weight of one or more NO-donating PGF2-alpha analogs (e.g., latanoprostene bunod).

### Prostaglandin Moieties

NO-donating PGF2-alpha analogs contain a chemical moiety that is an F-2-alpha prostaglandin. In certain embodiments of the pharmacological composition, non-limiting examples of the prostaglandin moiety of the NO-donating PGF2-alpha analog may include, but are not limited to, prostaglandin-F2-alpha, latanoprost, bimatoprost, travoprost, tafluprost or unoprotone, salts thereof or combinations thereof with the following structures:

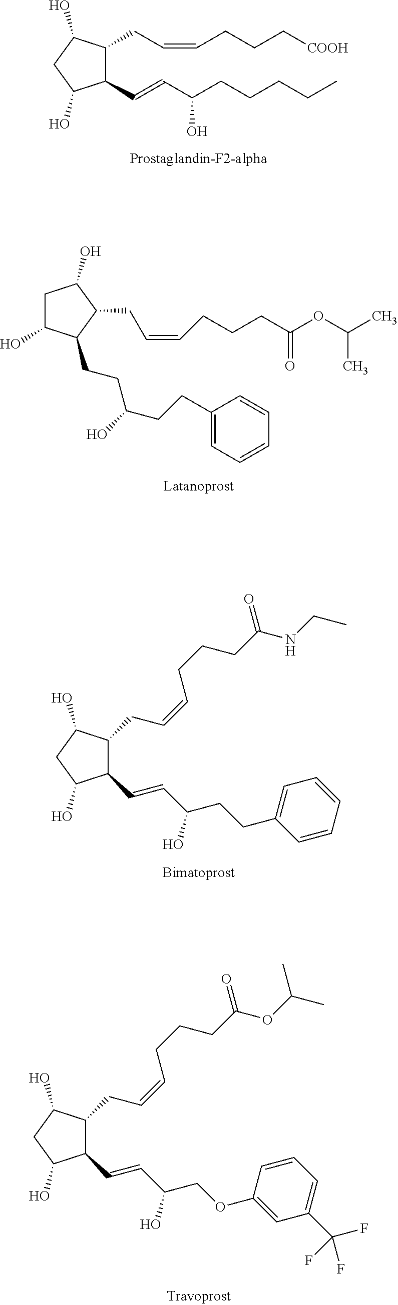 [SMILES: CCCCC[C@H](O)/C=C/[C@@H]1[C@@H](C/C=C\CCCC(=O)O)[C@@H](O)C[C@H]1O ; CC(C)OC(=O)CCC/C=C\C[C@@H]1[C@@H](CC[C@@H](O)CCc2ccccc2)[C@H](O)C[C@@H]1O ; CCNC(=O)CCC/C=C\C[C@@H]1[C@@H](/C=C/[C@@H](O)CCc2ccccc2)[C@H](O)C[C@@H]1O ; CC(C)OC(=O)CCC/C=C\C[C@@H]1[C@@H](/C=C/[C@@H](O)COc2cccc(C(F)(F)F)c2)[C@H](O)C[C@@H]1O]

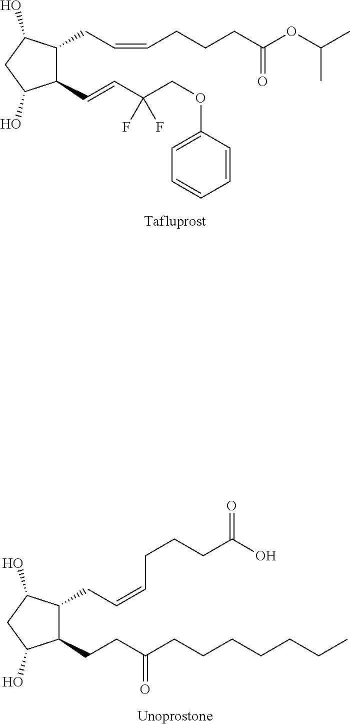 [SMILES: CC(C)OC(=O)CCC/C=C\C[C@@H]1[C@@H](/C=C/C(F)(F)COc2ccccc2)[C@H](O)C[C@@H]1O ; CCCCCCCC(=O)CC[C@@H]1[C@@H](C/C=C\CCCC(=O)O)[C@@H](O)C[C@H]1O]

In certain embodiments, the PGF2-alpha analog moiety is in a purified form. In certain embodiments, the purified PGF2-alpha analog moiety used in the present compositions and methods comprise (consist of, or consist essentially of) of the trans isomer.

The PGF2-alpha analog moiety may be chemically synthesized, or may be of plant, animal, or bacterial origin.

In certain embodiments, a PGF2-alpha analog moiety or derivative is administered in a therapeutically equivalent amount. In certain embodiments, a PGF2-alpha analog moiety or derivative is administered at a dose higher or lower than a therapeutically equivalent amount of PGF2-alpha analog moiety.

In certain embodiments, the present composition comprises about 0.005% to about 20% by weight, about 0.1% to about 18% by weight, about 0.2% to about 15% by weight, about 0.3% to about 10% by weight, about 0.5% to about 8% by weight, about 0.3%, about 0.4%, about 0.45%, about 0.5%, about 0.55% or about 0.6%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5% or about 10.0%, by weight of one or more PGF2-alpha analog moieties or derivatives.

Cutaneous Ulcers and/or Non-Healing Cutaneous Wounds

The present compositions and methods treat and/or prevent cutaneous ulcers and/or non-healing cutaneous wounds and/or a symptom associated therewith.

As used herein, the term “treatment” or “treating” refers to an amelioration, prophylaxis, or reversal of cutaneous ulcers and/or non-healing cutaneous wounds or a symptom associated therewith, for example, by lessening, or delaying the onset of, cutaneous ulcers and/or non-healing cutaneous wounds, or the symptom.

In certain embodiments, the cutaneous ulcer(s) and/or non-healing cutaneous wound(s) is/are secondary to microvascular compromise associated with type 1 or type 2 diabetes mellitus. The cutaneous ulcers and/or non-healing cutaneous wounds may or may not be increased from the baseline (before being treated by a topical NO-donating PGF2-alpha analog).

In certain embodiments, cutaneous ulcer(s) and/or non-healing cutaneous wound(s) is/are secondary to microvascular compromise associated with peripheral arterial disease.

In certain embodiments, cutaneous ulcer(s) and/or non-healing cutaneous wound(s) is/are secondary to microvascular compromise associated with autoimmune vasculopathy or vasculitis secondary to conditions that include, but are not limited to rheumatoid arthritis, systemic sclerosis, Reynaud disease, Reynaud syndrome, systemic lupus erythematosus, antiphospholipid syndrome, dermatomyositis, fibromyalgia, giant cell arteritis, gout, granulomatosis with polyangiitis, hyperimmunoglobulin D syndrome, inflammatory myopathy, Lyme disease, osteonecrosis, Paget's disease of bone, Kawasaki disease, polyarteritis nodosa, Takayasu's arteritis, eosinophilic granulomatosis with polyangiitis, microscopic polyangiitis, IgA vasculitis, Sjogren's syndrome, cryoglobulinemic vasculitis, drug-induced vasculitis, Behcet's disease, relapsing polychondritis and scleromalacia perforans.

In certain embodiments, cutaneous ulcer(s) and/or non-healing cutaneous wound(s) is/are secondary to microvascular compromise associated with sickle cell disease.

In certain embodiments, cutaneous ulcer(s) and/or non-healing cutaneous wound(s) is/are secondary to microvascular compromise associated with venous stasis of the back, abdomen, upper extremity or lower extremity or combinations thereof.

In certain embodiments, symptoms and signs associated with cutaneous ulcers and/or non-healing cutaneous wounds include, but are not limited to, epidermal breakdown, dermal breakdown, subcutaneous fat necrosis, local infection, systemic infection, bleeding, fever, chills, diaphoresis, dyspnea, purulent or non-purulent discharge, scabbing, scarring, osteomyelitis, flushing, skin-burning sensation, neuropathy, contact dermatitis.

In certain embodiments, cutaneous ulcers and/or non-healing cutaneous wounds involve the lower extremities, including the groin, thighs, knees, calves, ankles, feet and toes. Cutaneous ulcers and/or non-healing cutaneous wounds may affect other areas of the body, such as the upper extremities, including the shoulders, axilla, upper arms, forearms, wrists, hands and fingers; abdomen, back: face, involving cheeks, chin, nose, and/or forehead scalp, neck, ears, chest, back, and the eyes. In certain embodiments, cutaneous ulcers and/or non-healing cutaneous wounds are somatic.

Cutaneous ulcers and/or non-healing cutaneous wounds may refer to any skin or mucosal breakdown, or skin or mucosal irritation, or skin lesions. For example, cutaneous ulcers and/or non-healing cutaneous wounds may include dermatitis (e.g., radiodermatitis), eczema, epithelialysis, desquamation, redness, rubor, and/or rash. Cutaneous ulcers and/or non-healing cutaneous wounds may also comprise any type of cutaneous erythematous lesion, such as erythema ab igne (EAI), erythema chronicum migrans, erythema induratum, erythema infectiosum, erythema marginatum, erythema migrans, erythema multiforme, erythema nodosum, erythema toxicum, keratolytic winter erythema, palmar erythema, Stevens-Johnson syndrome, and toxic epidermal necrolysis (TEN, also known as Lyell's syndrome) and Naevus flammeus nuchae. Cutaneous ulcers and/or non-healing cutaneous wounds may be caused by or associated with irradiation, chemotherapy, and/or drug intake (e.g., antibiotics, barbituates, lamotrigine, phenytoin, nonsteroidal anti-inflammatory drugs (NSAIDs); or EGFR inhibitors). In an embodiment, cutaneous ulcers and/or non-healing cutaneous wounds is/are caused by allergen exposure, such as e.g. various allergens for allergy testing, urushiol, penicillin, latex, or wasp, fire ant and bee stings. In an embodiment, cutaneous ulcer(s) and/or non-healing cutaneous wound(s) is/are caused by fungal infection. In an embodiment, cutaneous ulcers and/or non-healing cutaneous wounds is/are caused by bacterial infection. In an embodiment, cutaneous ulcer(s) and/or non-healing cutaneous wound(s) is/are caused by viral infection. In an embodiment, the cutaneous ulcer(s) and/or non-healing cutaneous wound(s) is/are caused by a skin disease, which may be selected from the group consisting of psoriasis, atopic eczema or atopic dermatitis (neurodermatitis), eczema, or acne. In an embodiment, cutaneous ulcer(s) and/or non-healing cutaneous wound(s) is/are caused by a disease affecting internal or external mucosa, e.g. oral, nasal, or intestinal mucosa, and may be selected from the group consisting of inflammatory bowel disease, Morbus Crohn (or Crohn's disease), aphthous stomatitis, conjunctivitis, chronic obstructive pulmonary disease, peptic ulcers, alcohol abuse, and gastritis.

Cutaneous ulcers and/or non-healing cutaneous wounds may also be due to injections of biologic medications which are used to treat conditions such as rheumatoid arthritis, multiple sclerosis and other autoimmune diseases.

Cutaneous ulcers and/or non-healing cutaneous wounds may affect one or more layers of skin or mucosa, e.g. one or more layers of the epidermis and/or one or more layers of the dermis; or one or more layers of the mucous membranes, e.g. the mucosal epithelium (Lamina epithelialis mucosae) and/or the Lamina propria or the conjunctival or subconjunctival tissue.

In certain embodiments, the present compositions and methods treat and/or prevent cutaneous ulcers and/or non-healing cutaneous wounds. Cutaneous ulcers and/or non-healing cutaneous wounds may last about 30 minutes to about 4 weeks, about 1 hour to about 3 weeks, about 2 hours to about 2 weeks, about 2 hours to about 1 week, about 2 hours to about 6 days, about 2 hours to about 5 days, about 2 hours to about 4 days, about 2 hours to about 3 days, about 2 hours to about 48 hours, about 3 hours to about 36 hours, about 4 hours to about 30 hours, about 5 hours to about 24 hours, about 6 hours to about 24 hours, about 7 hours to about 24 hours, about 1 week to about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, or about 24 weeks, about 1 month to about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, or about 24 months.

In one embodiment, the cutaneous ulcer(s) and/or non-healing cutaneous wound(s) may temporally resolve with reapplication of NO-donating PGF2-alpha analog(s). However, the cutaneous ulcer(s) and/or non-healing cutaneous wound(s) may quickly return and may become progressively worse with repeated applications of topical NO-donating PGF2-alpha analog(s). In one embodiment, the cutaneous ulcer(s) and/or non-healing cutaneous wound(s) resolve over several days after topical NO-donating PGF2-alpha analog(s) is/are discontinued.

Cutaneous ulcer(s) and/or non-healing cutaneous wound(s) or a symptom associated therewith encompasses different degrees or grades of severity or a symptom associated therewith, from mild to severe.

In view of the present disclosure, a skin area that is affected by cutaneous ulcer(s) and/or non-healing cutaneous wound(s) or that is prone to be affected by cutaneous ulcer(s) and/or non-healing cutaneous wound(s) can be identified using any diagnostic signs or means known in the art, and can be treated by methods according to the present composition and method.

The present compositions and methods may elicit a marked decrease in cutaneous ulcer(s) and/or non-healing cutaneous wound(s), or complete disappearance of cutaneous ulcer(s) and/or non-healing cutaneous wound(s). For example, the present compositions and methods may result in significant restoration of the skin integrity to that before the occurrence of cutaneous ulcer(s) and/or non-healing cutaneous wound(s) due to administration of a topical NO-donating PGF2-alpha analog(s).

In certain embodiments, the skin area (e.g., affected by cutaneous ulcer(s) and/or non-healing cutaneous wound(s) or on which a topical NO-donating PGF2-alpha analog is applied) returns to its baseline level between applications of the present agents or present compositions.

Cutaneous ulcers and/or non-healing cutaneous wounds or a symptom associated therewith may be assessed by any suitable systems.

For example, cutaneous ulcers and/or non-healing cutaneous wounds or a symptom associated therewith can be rated by a clinician based on ulceration and/or wound severity values listed in “The Society for vascular surgery lower extremity threatened limb classification system: Risk stratification based on wound, ischemia and foot infection” (Mills, et al, 2013).

The efficacy of the treatment can be measured using method known in the art. For example, the efficacy can be measured by the grades of improvement in ulceration and/or wound severity values listed in the classification system presented in “The Society for vascular surgery lower extremity threatened limb classification system: Risk stratification based on wound, ischemia and foot infection” (Mills, et al, 2013).

In certain embodiments, the noticeable effect may progress to maximum improvement for a sustained period of time. The maximum improvement may then decline to noticeable effect, which may then disappear.

In certain embodiments, the present composition and method result in significantly more effective treatment of cutaneous ulcers and/or non-healing cutaneous wounds and the symptoms thereof than a vehicle control.

In certain embodiments, the present composition and method result in significantly more reduction of cutaneous ulcers and/or non-healing cutaneous wounds compared to a vehicle control as measured by skin ulceration and/or wound severity values listed in the classification system as presented in “The Society for vascular surgery lower extremity threatened limb classification system: Risk stratification based on wound, ischemia and foot infection” (Mills, et al, 2013).

Cutaneous ulcers and/or non-healing cutaneous wounds at various time points may be assessed. Cutaneous ulcers and/or non-healing cutaneous wounds may be evaluated at various time points before and after the start of treatment by the present composition (e.g., comprising a topical NO-donating PGF2-alpha analog). In certain embodiments of the present methods, cutaneous ulcers and/or non-healing cutaneous wounds may be assessed prior to the start of treatment by a topical NO-donating PGF2-alpha analog, after the start of treatment by a topical NO-donating PGF2-alpha analog, before the start of treatment by the present composition (e.g., comprising a topical NO-donating PGF2-alpha analog), and/or after the start of treatment by the present composition (e.g., a topical NO-donating PGF2-alpha analog).

A baseline severity value of the skin ulceration and/or wound may be obtained prior to the start of treatment by a topical NO-donating PGF2-alpha analog, or at the beginning of treatment by a topical NO-donating PGF2-alpha analog (which may be a first treatment in a course of several subsequent treatments). Cutaneous ulcers and/or non-healing cutaneous wounds may be assessed at two or more time-points prior to treatment a topical NO-donating PGF2-alpha analog, and an according mean baseline severity value may be calculated.

After the start of treatment by a topical NO-donating PGF2-alpha analog and before the start of treatment by the present composition (e.g., comprising a topical NO-donating PGF2-alpha analog such as latanoprostene bunod), skin ulceration and/or wound severity values may be obtained at 1, 2, 3, 4, 5, 6, 7, 8 or more time-points. The one or more severity values may be defined as initial severity values.

The gradient between two or more of said skin ulceration and/or wound severity values may be determined, e.g. between two or more subsequent measurements or skin ulceration and/or wound severity values, or between two or more measurements or skin ulceration and/or wound severity values over treatment or observation time. An increase of the skin ulceration and/or wound severity value between two or more skin ulceration and/or wound severity values over time indicates progression of skin ulceration and/or wound severity. No significant change of the skin ulceration and/or wound severity value between two or more skin ulceration and/or wound severity values over time indicates a stable skin or mucosal condition, is effective in preventing skin ulceration and/or wound severity progression, is not effective in treating skin ulceration and/or wound severity, and/or is not effective in ameliorating the appearance of skin ulceration and/or wound severity. A decrease of the skin ulceration and/or wound severity value between two or more skin ulceration and/or wound severity values over time indicates regression of skin ulceration and/or wound severity, i.e., that the composition is effective in treating skin ulceration and/or wound severity, or in ameliorating the appearance of skin ulceration and/or wound severity.

In one embodiment, the skin or mucosal area is a skin area. In another embodiment, the skin or mucosal area is a mucosal area. The mucosal area may be an external or internal mucosa, e.g. nasal, oral, intestinal mucosa.

In certain embodiments, the skin or mucosal area is a gross area comprising one or more segment areas. The skin ulceration and/or wound severity value may be calculated separately for each subarea. If the skin ulceration and/or wound severity value of a skin or mucosal area is measured by obtaining an image, a subarea may also be a single pixel of the image, i.e., the skin ulceration and/or wound severity value is calculated for one or more single pixels. Accordingly, a mean skin ulceration and/or wound severity value may be calculated of one or more single pixels of the image, the gross area, and/or the segment area.

The segment area may contain a cutaneous ulcer(s) and/or non-healing cutaneous wound(s) (or a representative part of cutaneous ulcer(s) and/or non-healing cutaneous wound(s)). In one embodiment, the area of cutaneous ulcer(s) and/or non-healing cutaneous wound(s) is an area prone to development of cutaneous ulcer(s) and/or non-healing cutaneous wound(s), which may e.g. be caused by a disease and/or treatment as described herein. In another embodiment, the area of cutaneous ulcer(s) and/or non-healing cutaneous wound(s) is an area characterized by existing cutaneous ulcer(s) and/or non-healing cutaneous wound(s). The treatment may be local (topical) or systemic. Accordingly, in an embodiment, the segment area prone to cutaneous ulcer(s) and/or non-healing cutaneous wound(s) or characterized by cutaneous ulcer(s) and/or non-healing cutaneous wound(s) may be the analyzed treatment area of a local treatment, or the analyzed area at which the cutaneous ulcer(s) and/or non-healing cutaneous wound(s) occur(s) upon systemic treatment. The analyzed segment area may also be a treatment area (or a representative part of a treatment area), i.e. an analyzed area that is or will be treated, e.g. with the present composition. In one embodiment, the analyzed wound area is also a treatment area.

The analyzed segment area may also be a reference area (or a representative part of a reference area). The reference area may be an area of the same subject. In an embodiment, the reference area may be an area of one or more subjects other than the subject to be assessed. Said different subjects may be of the same race, of the same or similar skin color, and/or of the same skin type (e.g., according to the Fitzpatrick Skin Scale). In an embodiment, these different subjects have the same kind of skin condition, e.g., cutaneous ulcer(s) and/or non-healing cutaneous wound(s). In still another embodiment, these different subjects suffer from the same or a similar disease and/or undergo the same or similar treatment. Accordingly, a reference curve of two or more reference skin ulceration and/or wound severity values may be generated for comparison to the subject's measurements. In general, for any comparison of skin ulceration and/or wound severity values (e.g., to reference values), the area under the curve between two or more skin ulceration and/or wound severity values may be determined and compared to, e.g., the area under the curve of two or more reference skin ulceration and/or wound severity values.

The reference area may be an area similar to the treatment area, i.e. an area of the same or a similar region of the body, and/or of the same or similar nature (e.g. of similar color and/or shape). In one embodiment, the reference area is adjacent to the segment area to be compared to, e.g. the treatment area. In one embodiment, the reference area is of the same size as the segment area to be compared to, e.g. treatment area. The skin ulceration and/or wound severity value calculated for a reference area may also be called reference skin ulceration and/or wound severity value.

In one embodiment, the reference area is not characterized by cutaneous ulcers and/or non-healing cutaneous wounds, i.e., is free from cutaneous ulcers and/or non-healing cutaneous wounds. For example, the reference area may be the treatment area prior to treatment with a topical NO-donating PGF2-alpha analog, thus, prior to development of cutaneous ulcers and/or non-healing cutaneous wounds. The reference area may be the treatment area prior to treatment with the present composition. In one embodiment, the reference area is not a treatment area. In an example, the reference area is left completely untreated. In a further embodiment, the reference area is treated with a placebo or with a reference treatment, such as, the gold standard treatment or a comparative product. In another embodiment, the reference area is characterized by a cutaneous ulcer(s) and/or non-healing cutaneous wound(s), as the present composition and method can ameliorate. In another embodiment, the reference area is an area of cutaneous ulcers and/or non-healing cutaneous wounds and/or treatment area prior to development of cutaneous ulcers and/or non-healing cutaneous wounds and/or prior to treatment. The reference skin ulceration and/or wound severity value calculated for a reference area that is an area containing cutaneous ulcers and/or non-healing cutaneous wounds and/or a treatment area, but based on a measurement prior to development of cutaneous ulcers and/or non-healing cutaneous wounds and/or prior to treatment, may also be called baseline skin ulceration and/or wound severity value or initial skin ulceration and/or wound severity value.

In an embodiment, the skin ulceration and/or wound severity value is compared to a reference skin ulceration and/or wound severity value. For example, the skin ulceration and/or wound severity value of a segment area (e.g. a treatment and/or erythema area) is compared to one or more reference skin ulceration and/or wound severity values. In one embodiment, the reference skin ulceration and/or wound severity value is calculated based on one or more reference areas. In another embodiment, the reference skin ulceration and/or wound severity value is the skin ulceration and/or wound severity value of the same segment area from which the follow-up skin ulceration and/or wound severity value is calculated, e.g. an area containing a cutaneous ulcer(s) and/or non-healing cutaneous wound(s) and/or treatment area, assessed prior to treatment and/or development of a cutaneous ulcer(s) and/or non-healing cutaneous wound(s).

In another embodiment, the skin ulceration and/or wound severity value may be compared to more than one reference skin ulceration and/or wound severity values and/or one or more reference gradients between two or more skin ulceration and/or wound severity values of reference areas. Said reference skin ulceration and/or wound severity values and/or reference gradients may include reference skin ulceration and/or wound severity values of the same subject or of one or more different subjects. For example, a reference skin ulceration and/or wound severity gradient, or a rating or reference curve may be determined from subjects with the same or similar type and/or grade, e.g. with the same disease or treatment. Accordingly, the severity of a subject may be assessed by calculating one or more skin ulceration and/or wound severity values and comparing the one or more skin ulceration and/or wound severity values to one or more reference skin ulceration and/or wound severity values, a reference skin ulceration and/or wound severity gradient between two or more reference skin ulceration and/or wound severity values, and/or to a rating or reference curve.

The measurement or image may be obtained by any suitable measurement method or imaging method, such as e.g. spectrophotometry, video, video frame buffer, and/or photography. For example, the measurement or image may be obtained by a method not requiring any direct contact with the gross area and/or segment areas, e.g. photography. In an embodiment, the measurement or image is obtained by spectrophotometry and/or photography. In an embodiment, the measurement or image is obtained by digital photography. The measurement or image may also be obtained by using endoscopic devices. In one embodiment, the skin or mucosal area is an internal mucosal area and the light reflectance is measured by endoscopic methods.

For example, the skin ulceration and/or wound severity value may be converted into a relative value compared to the respective maximal value, which is set to 100%, in order to compare several skin ulceration and/or wound severity values based on different measurement methods or imaging methods, and/or based on different color depths.

In one embodiment, the methods of the invention are repeated at several time-points, e.g. at one or more time-points prior to, during, and/or after treatment, development, and/or amelioration of a cutaneous ulcer(s) and/or non-healing cutaneous wound(s).

In particular, the light reflectance of a skin or mucosal area of a subject may be measured at one or more time-points prior to, during, and/or after development or progression of a cutaneous ulcer(s) and/or non-healing cutaneous wound(s), or prior to, during, and/or after amelioration or regression of a cutaneous ulcer(s) and/or non-healing cutaneous wound(s), or prior to, during, and/or after the period of administration of the pharmaceutical preparation, or prior to, during, and/or after the period of treatment (e.g. a local treatment of the skin or mucosal area, or a systemic treatment of the subject). The gradient between two or more of said skin ulceration and/or wound severity values may be determined, e.g. between two or more subsequent measurements or skin ulceration and/or wound severity values, or between two or more measurements or skin ulceration and/or wound severity values over treatment or observation time. Accordingly, the skin ulceration and/or wound severity value calculated based on a measurement taken prior to any treatment (e.g. radiation) and/or manifestation of a cutaneous ulcer(s) and/or non-healing cutaneous wound(s) may be the baseline skin ulceration and/or wound severity value. Any skin ulceration and/or wound severity value calculated based on a measurement taken during or after any treatment (e.g. radiation) and/or manifestation of a cutaneous ulcer(s) and/or non-healing cutaneous wound(s) may be a follow-up skin ulceration and/or wound severity value.

Combination with Other Active Agents or Treatments

The present agent (e.g., a topical NO-donating PGF2-alpha analog) or composition may be administered to the subject simultaneously with, before, after, or in a sequence and within a time interval of, the administration of another separate treating agent or treatment procedure to be described herein, such that the present agent or composition can act together to treat or prevent cutaneous ulcers and/or non-healing cutaneous wounds and symptoms associated therewith (e.g., a cutaneous ulcer(s) and/or non-healing cutaneous wound(s). For example, the present agent (e.g., a topical NO-donating PGF2-alpha analog) or composition and a another separate treating agent or treatment procedure can be administered in the same or separate formulations at the same time or different times. In certain embodiments, the present agent (e.g., a topical NO-donating PGF2-alpha analog) or composition can be administered before or after the administration of another separate treating agent or treatment procedure.

By co-administration it is meant either the administration of a single composition containing both the present agent (e.g., a topical NO-donating PGF2-alpha analog) another separate treating agent or treatment procedure to be described herein, or the administration of the present agent (e.g., a topical NO-donating PGF2-alpha analog) and another separate treating agent or treatment procedure to be described herein as separate compositions within short enough time periods, with no particular order.

The present agent (e.g., a topical NO-donating PGF2-alpha analog) can be combined and administered with a another separate treating agent or treatment procedure to be described herein in separate compositions or treatment sessions. In certain embodiments, the separate compositions or treatments are administered simultaneously. In certain embodiments, the separate compositions or treatments are not administered simultaneously, such as, for example, in a sequential manner.

The present compound or composition may be administered to a subject alone, or may be administered to a subject in combination with one or more other treatments/agents (a second agent).

In certain embodiments, the second agent is a local anesthetic, a nonsteroidal anti-inflammatory agents (NSAID), an antihistamine, an antibiotic, a nitric oxide synthase (NOS) activator or up-regulator, a corticosteroid, acetaminophen, a vasodilator, or combinations thereof.

In certain embodiments, the present compound or composition is administered to a subject in combination with one or more treatments/agents such as corticosteroids, NSAID's, an stimulator of nitric oxide synthesis, an opioid agonists and/or antagonists.

In certain embodiments, the present compound or composition is administered to a subject in combination with one or more treatments/agents such as a capsaicinoid (e.g. capsaicin), a topical H-1 histamine receptor antagonist, a mast cell stabilizer (e.g., olopatadine etc.), an antibacterial agent, an anthelmintic agent, an anti-fungal agent, a pro-angiogenesis agent (e.g. vascular endothelial growth factor (VEGF)), a skin growth factor (e.g. epidermal growth factor (EGF)), a steroidal anti-inflammatory agent, a non-steroidal anti-inflammatory agent, or antioxidant or, as well as halogens.

In some embodiments, the second agent is one or more nonsteroidal anti-inflammatory agents (NSAIDs). NSAIDs include, but are not limited to, salicylates such as aspirin (acetylsalicylic acid), diflunisal, salsalate, salicylic acid, acetyl salicylate, methyl salicylate, methyl acetylsalicylate, trolamine salicylate and lysine salicylate; p-amino phenol derivatives such as paracetamol and phenacetin; propionic acid derivatives such as ibuprofen, naproxen, fenoprofen, ketoprofen, dexketoprofen, flurbiprofen, oxaprozin and loxoprofen; acetic acid derivatives such as indomethacin, sulindac, etodolac, ketorolac, diclofenac and nabumetone; enolic acid (oxicam) derivatives such as piroxicam, meloxicam, tenoxicam, droxicam, lornoxicam and isoxicam; and fenamic acid derivatives (fenamates) such as mefenamic acid, meclofenamic acid, flufenamic acid and tolfenamic acid.

In some embodiments, the second agent is one or more corticosteroids. Corticosteroids include, but are not limited to alclometasone dipropionate, amcinonide, amcinafel, amcinafide, beclamethasone, betamethasone, betamethasone dipropionate, betamethasone valerate, budesonide, clobetasone propionate, chloroprednisone, clocortelone, cortisol, cortisone, cortodoxone, difluorosone diacetate, descinolone, desonide, defluprednate, dihydroxycortisone, desoximetasone, dexamethasone, deflazacort, diflorasone diacetate, dichlorisone, esters of betamethasone, flucetonide, flucloronide, fluorocortisone, flumethasone, flunisolide, fluocinonide, fluocinolone acetonide, flucortolone, fluperolone, fluprednisolone, fluroandrenolone acetonide, fluocinolone acetonide, flurandrenolide, fluorametholone, fluticasone propionate, hydrocortisone, hydrocortisone butyrate, hydrocortisone valerate, hydrocortamate, medrysone, meprednisone, methylprednisone, methylprednisolone, mometasone furoate, paramethasone, prednisone, prednisolone, prednidone, triamcinolone acetonide, and triamcinolone.

In some embodiments, the second agent is one or more opioid agonists and/or antagonists. Examples of opioid agonists/antagonists include but are not limited to purified alkaloids of opium consisting of phenanthrenes and benzylisoquinolines, semi-synthetic derivatives of morphine, phenylpiperidine derivatives, morphinan derivatives, benzomorphan derivatives, diphenyl-heptane derivatives, and propionanilide derivatives.

In some embodiments, the second agent is an inflammation mediator antagonist. Exemplary inflammation mediator antagonists include, but are not limited to, diethylenediamine derivatives such as cinnarizine and cyclizine; aminopropane derivatives (dexchlorpheniramine, triprolidine); phenothiazine derivatives (alimemazine, promethazine); auranofin; lisophyline; A802715; sulfasalazine; cetirizine HCl; loratidine; esbatine; setastine HCl.

Non-limiting examples of compounds that can be administered in combination with the present compound or composition include, (a) agents which modify skin differentiation and/or proliferation and/or pigmentation, such as retinoic acid and isomers thereof, retinol and its esters, vitamin D and derivatives thereof, estrogens such as estradiol, kojic acid or hydroquinone; (b) antibacterial agents such as clindamycin phosphate, erythromycin or antibiotics of the tetracycline family; (c) antiparasitic agents, such as metronidazole, crotamiton or pyrethroids; (d) antifungal agents, such as compounds belonging to the imidazole family (e.g., econazole, ketoconazole or miconazole or salts thereof), polyene compounds, such as amphotericin B, compounds of the allylamine family such as terbinafine; (e) steroidal anti-inflammatory agents, such as hydrocortisone, betamethasone valerate or clobetasol propionate, or nonsteroidal anti-inflammatory agents, such as ibuprofen and its salts, diclofenac and its salts, acetylsalicylic acid, acetaminophen or glycyrrhetinic acid; (f) anesthetics such as lidocaine hydrochloride and derivatives thereof; (g) antipruriginous agents such as thenaldine or trimeprazine; (h) antiviral agents such as acyclovir; (i) keratolytic agents such as -alpha- and beta-hydroxycarboxylic acids or beta-ketocarboxylic acids, their salts, amides or esters and more particularly hydroxy acids such as glycolic acid, lactic acid, salicylic acid, citric acid and fruit acids in general, and derivatives of salicylic acid such as 5-n-octanoylsalicylic acid; (j) anti-free-radical agents, such as -alpha-tocopherol or its esters, superoxide dismutases, certain metal-chelating agents or ascorbic acid and its esters; (k) antiseborrhoeic agents such as progesterone; (l) antidandruff agents such as octopirox or zinc pyrithione; (m) antiacne agents such as retinoic acid or benzoyl peroxide.

In some embodiments, the second agent is gabapentin, pregabalin, menthol, boswellic acid, DMSO, methyl sulfonylmethan, emu oil, hyaluronic acid, santalol, santalyl acetate, amyris alcohol, amyris acetate, or combinations thereof. U.S. Patent Publication No. 20140134261.

In certain embodiments, the topical NO-donating PGF2-alpha analog (e.g. latanoprostene bunod) is combined with a second, or adjunctive treatment consisting of hyperbaric oxygen therapy.

In certain embodiments, the topical NO-donating PGF2-alpha analog (e.g. latanoprostene bunod) is combined with a second, or adjunctive treatment consisting of microneedling therapy.

In certain embodiments, the topical NO-donating PGF2-alpha analog (e.g. latanoprostene bunod) is combined with a second, or adjunctive treatment consisting of low-energy extracorporeal shock wave (LE-ESW) treatment.

In certain embodiments, the topical NO-donating PGF2-alpha analog (e.g. latanoprostene bunod) is combined with a second, or adjunctive treatment consisting of low-intensity cathodal direct current therapy.

In certain embodiments, combination therapy means simultaneous administration of the compounds in the same composition, simultaneous administration of the compounds in separate compositions, or separate administration of the compounds (in separate compositions).

In certain embodiments, the second agent/treatment is used as adjunctive therapy to the present compound or composition. In certain embodiments, the treatment includes a phase wherein treatment with the second agent/treatment takes place after treatment with the present compound or composition has ceased. In certain embodiments, the treatment includes a phase where treatment with the present compound or composition and treatment with the second agent/treatment overlap.

Combination therapy can be sequential or can be administered simultaneously. In either case, these drugs and/or therapies are said to be “co-administered.” It is to be understood that “co-administered” does not necessarily mean that the drugs and/or therapies are administered in a combined form (i.e., they may be administered separately (e.g., as separate compositions or formulations) or together (e.g., in the same formulation or composition) to the same or different sites at the same or different times).

In certain embodiments, a subject is treated concurrently (or concomitantly) with the present compound or composition and a second agent. In certain embodiments, a subject is treated initially with the present compound or composition, followed by cessation of the present compound or composition treatment and initiation of treatment with a second agent. In certain embodiments, the present compound or composition is used as an initial treatment, e.g., by administration of one, two or three doses, and a second agent is administered to prolong the effect of the present compound or composition, or alternatively, to boost the effect of the present compound or composition. A person of ordinary skill in the art will recognize that other variations of the presented schemes are possible, e.g., initiating treatment of a subject with the present compound or composition, followed by a period wherein the subject is treated with a second agent as adjunct therapy to the present compound or composition treatment, followed by cessation of the present compound or composition treatment.

The present compound and the other pharmaceutically active agent(s) may be administered together or separately and, when administered separately this may occur simultaneously or sequentially in any order. The amounts of the present compound and the other pharmaceutically active agent(s) and the relative timings of administration will be selected in order to achieve the desired combined therapeutic effect.

In various embodiments, the therapies (e.g., a composition provided herein and a second agent in a combination therapy) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In certain embodiments, the therapies are administered no more than 24 hours apart or no more than 48 hours apart. In certain embodiments, two or more therapies are administered within the same patient visit. In other embodiments, the composition provided herein and the second agent are administered concurrently. In other embodiments, the composition provided herein and the second agent are administered at about 2 to 4 days apart, at about 4 to 6 days apart, at about 1 week part, at about 1 to 2 weeks apart, or more than 2 weeks apart. In certain embodiments, administration of the same agent may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same agent may be repeated and the administration may be separated by at least at least 1 day, 2 days, 3 days, 5 days, 10 days, 15 days, 30 days, 45 days, 2 months, 75 days, 3 months, or 6 months. In certain embodiments, a composition provided herein and a second agent are administered to a subject in a sequence and within a time interval such that the composition provided herein can act together with the other agent to provide an increased benefit than if they were administered otherwise. For example, the second active agent can be administered at the same time or sequentially in any order at different points in time; however, if not administered at the same time, they should be administered sufficiently close in time so as to provide the desired therapeutic or prophylactic effect. In one embodiment, the composition provided herein and the second active agent exerts their effect at times which overlap. Each second active agent can be administered separately, in any appropriate form and by any suitable route. In other embodiments, the composition provided herein is administered before, concurrently or after administration of the second active agent. In other embodiments, courses of treatment are administered concurrently to a patient, i.e., individual doses of the second agent are administered separately yet within a time interval such that the compound provided herein can work together with the second active agent. For example, one component can be administered once per week in combination with the other components that can be administered once every two weeks or once every three weeks. In other words, the dosing regimens are carried out concurrently even if the therapeutics are not administered simultaneously or during the same day.

The second agent can act additively or synergistically with the present agent/compound. In one embodiment, the composition provided herein is administered concurrently with one or more second agents in the same pharmaceutical composition. In another embodiment, a composition provided herein is administered concurrently with one or more second agents in separate pharmaceutical compositions. In still another embodiment, a composition provided herein is administered prior to or subsequent to administration of a second agent. Also contemplated are administration of a composition provided herein and a second agent by the same or different routes of administration, e.g., oral and parenteral. In certain embodiments, when the composition provided herein is administered concurrently with a second agent that potentially produces adverse side effects including, but not limited to, toxicity, the second active agent can advantageously be administered at a dose that falls below the threshold that the adverse side effect is elicited.

Encompassed by the present disclosure are methods to treat a human subject experiencing the signs and symptoms of a cutaneous ulcer(s) and/or non-healing cutaneous wound(s). In certain embodiments, the present method treats, cures, prevents or delays a cutaneous ulcer(s) and/or non-healing cutaneous wound(s) in a subject.

### Dosages

The present agent (such as a topical NO-donating PGF2-alpha analog including latanoprostene bunod or a derivative, analogue, or salt thereof, and other compounds as described herein) may be present in the present composition in an amount ranging from about 0.001 wt % to about 5 wt %, from about 0.001 wt % to about 2 wt %, from about 0.001 wt % to about 1.5 wt %, from about 0.001 wt % to about 1 wt %, from about 0.001 wt % to about 0.8 wt %, from about 0.001 wt % to about 0.5 wt %, from about 0.002 wt % to about 0.3 wt %, from about 0.005 wt % to about 0.2 wt %, from about 0.005 wt % to about 0.1 wt %, from about 0.005 wt % to about 0.08 wt %, from about 0.005 wt % to about 0.05 wt %, from about 0.005 wt % to about 0.04 wt %, from about 0.005 wt % to about 0.03 wt %, from about 0.005 wt % to about 0.02 wt %, from about 0.01 wt % to about 0.08 wt %, from about 0.01 wt % to about 0.075 wt %, from about 0.01 wt % to about 0.06 wt %, from about 0.025 wt % to about 0.08 wt %, from about 0.025 wt % to about 0.075 wt %, from about 0.01 wt % to about 0.05 wt %, from about 0.01 wt % to about 0.04 wt %, from about 0.01 wt % to about 0.03 wt %, from about 0.01 wt % to about 0.02 wt %, from about 0.01 wt % to about 0.025 wt %, about 0.005 wt %, about 0.006 wt %, about 0.007 wt %, about 0.008 wt %, about 0.009 wt %, about 0.01 wt %, about 0.011 wt %, about 0.012 wt %, about 0.013 wt %, about 0.014 wt %, about 0.015 wt %, about 0.016 wt %, about 0.017 wt %, about 0.018 wt %, about 0.019 wt %, about 0.02 wt %, or about 0.025 wt %, of the total weight of the composition. The present agent (such as a topical NO-donating PGF2-alpha analog including latanoprostene bunod or a derivative, analogue, or salt thereof, and other compounds as described herein) may be present in the present composition in an amount ranging from 0.20-30% by weight, 0.075-30% by weight, 0.2-30%, or 2-20%, 2-10%, 5-15%, 0.075-30 wt. %, 0.2 wt % to 30 wt %, between 1 wt % and 20 wt %, e.g. 1 wt %, 5 wt %, 10 wt %, and 20 wt %, from about 10% to about 50% by weight, from about 10% to about 40% by weight, from about 10% to about 30% by weight, from about 10 to about 25% by weight, from about 10% to about 20% by weight, or from about 10% to about 15% by weight, of the total weight of the composition.

When a NO-donating PGF2-alpha analog other than latanoprostene bunod is used in the present composition, since potency can vary, the amount of the NO-donating PGF2-alpha analog in the composition may be the amount which achieves the same or similar results achieved by the weight percent ranges for latanoprostene bunod by about 10-fold more or less, 20-fold more or less, 30-fold more or less, 40-fold more or less, 50-fold more or less, 60-fold more or less, 70-fold more or less, 80-fold more or less, 100-fold more or less, at least 100-fold more or less, 120-fold more or less, 150-fold more or less, or 200-fold more or less, than a dose of latanoprostene bunod.

As used herein, “wt %” refers to % w/w of the composition.

In certain embodiments, the amount of a topical formulation applied to the affected skin area ranges from about 0.01 g/cm² of skin surface area to about 5 g/cm², from 0.2 g/cm² to about 0.5 g/cm², from about 0.0001 g/cm² to about 0.05 g/cm², from about 0.0001 g/cm² to about 0.01 g/cm², from about 0.001 g/cm² to about 0.003 g/cm², or from 0.002 g/cm² to about 0.005 g/cm² of skin surface area.

The present composition may be administered once, twice, three times, four times, five times, six times or more per day, or as needed, during the course of treatment. In certain embodiments, the present agent/composition is administered at least once a day, at least twice a day, at least three times per day, or more. In certain embodiments, the present agent/composition is administered at least once a week, at least twice a week, at least three times a week, at least once per month, at least twice per month, or more frequently. Treatment can continue as long as needed. In one embodiment, the topical composition is topically applied to the affected skin area once daily.

The present composition may be administered daily, weekly, biweekly, several times daily, semi-weekly, every other day, bi-weekly, quarterly, several times per week, semi-weekly, monthly etc., to maintain an effective dosage level. The duration and frequency of treatment may depend upon the subject's response to treatment.

In certain embodiments, a subject may be administered 1 dose, 2 doses, 3 doses, 4 doses, 5 doses, 6 doses or more of the present composition. In certain embodiments, a single dose of the present agent/composition is administered in the present method. In certain embodiments, multiple doses of the present agent/composition (e.g., 2 doses, 3 doses, 4 doses, 5 doses, 6 doses, 7 doses, 8 doses, 9 doses, 10 doses or more) are administered in the present method.

In certain embodiments, the administration of the present agent/composition is continued over a period of up to 2 days, up to 3 days, up to 4 days, up to 5 days, up to 6 days, up to 1 week, up to 2 weeks, up to 3 weeks, up to 4 weeks, 2 weeks, 3 weeks, 4 weeks, 5 weeks, 6 weeks, 7 weeks, 8 weeks, 9 weeks, 10 weeks, or longer.

In certain embodiments, the present agent/composition is administered once, twice, at least twice, at least three times, at least four times, at least five time, at least six times, at least seven times, at least eight times, at least nine times, or more per treatment.

### Subjects

The subject may be a human. In an embodiment, the subject is suffering from a cutaneous ulcer(s) and/or non-healing cutaneous wound(s) and is or will be treated with a treatment that may treat a cutaneous ulcer(s) and/or non-healing cutaneous wound(s) and/or ameliorate the appearance of a cutaneous ulcer(s) and/or non-healing cutaneous wound(s). In an embodiment, the subject is suffering from a disease that compromises microvascular circulation and skin integrity, which may be selected from the group consisting of type 1 diabetes mellitus, type 2 diabetes mellitus, peripheral arterial disease, systemic lupus erythematosus, sarcoidosis, sickle cell disease, sickle cell trait, venous stasis, rheumatoid arthritis, systemic sclerosis, Reynaud disease, Reynaud syndrome, systemic lupus erythematosus, antiphospholipid syndrome, dermatomyositis, fibromyalgia, giant cell arteritis, gout, granulomatosis with polyangiitis, hyperimmunoglobulin D syndrome, inflammatory myopathy, Lyme disease, osteonecrosis, Paget's disease of bone, Kawasaki disease, polyarteritis nodosa, Takayasu's arteritis, eosinophilic granulomatosis with polyangiitis, microscopic polyangiitis, IgA vasculitis, Sjogren's syndrome, cryoglobulinemic vasculitis, drug-induced vasculitis, Behcet's disease, relapsing polychondritis and scleromalacia perforans In an embodiment, the subject is suffering from a disease affecting internal or external mucosa, e.g. oral, nasal, or intestinal mucosa. For example, the subject is suffering from inflammatory bowel disease, Morbus Crohn (or Crohn's disease), aphthous stomatitis, conjunctivitis, chronic obstructive pulmonary disease, peptic ulcers, alcohol abuse, or gastritis.

### Kits

The present disclosure also encompasses an article of manufacture, e.g., a kit. The article of manufacture may contain the present composition in a suitable container with labeling and instructions for use. In certain embodiments, the container can be a dropper or tube with a suitable small orifice size, such as an extended tip tube made of any pharmaceutically suitable material. The topical formulations can be filled and packaged into a plastic squeeze bottle or tube. Optionally, an applicator can be provided in or attached to the container, or separately from the container.

Instructions may be packaged with the composition, for example, a pamphlet or package label. The labeling instructions explain how to the present composition, in an amount and for a period of time sufficient to treat or prevent cutaneous ulcers and/or non-healing cutaneous wounds and their symptoms. In certain embodiments, the label includes the dosage and administration instructions, the topical formulation's composition, the clinical pharmacology, drug resistance, pharmacokinetics, absorption, bioavailability, and/or contraindications.

### Topical Administration

In certain embodiments, the present composition is formulated for topical administration. The terms “topically administrable composition,” a “topical composition,” or a “topical formulation,” as used herein, refer to any formulation or composition which is pharmaceutically and/or cosmetically acceptable for topical delivery of the specified compounds according to embodiments of the invention. The composition may be administered to a defined area of the body such as a defined area of skin surface or mucous membrane.

The present composition may additional contain a physiologically acceptable medium, such as a vehicle and/or a carrier. By “physiologically acceptable medium” is intended a cosmetically and/or dermatologically acceptable medium, which is compatible with the skin.

In some embodiments, the present composition can additionally include one or more pharmaceutically acceptable excipients. One of ordinary skill in the art would be familiar with pharmaceutically acceptable excipients. For example, the pharmaceutically acceptable excipient may be a water soluble sugar, such as mannitol, sorbitol, fructose, glucose, lactose, and sucrose.

The present composition can be formulated in any pharmaceutical form normally provided for topical application to the skin, in particular formulated as solutions or dispersions of lotion or serum type, emulsions of liquid or semi-liquid consistency of the milk type, obtained by dispersion of a fatty phase in an aqueous phase (O/W) or, conversely, (W/O), or suspensions or emulsions of soft consistency of the aqueous or anhydrous cream or gel type, or, alternatively, microgranules, nanoparticles, microemulsions, nano-capsules, or vesicle dispersions of ionic and/or nonionic type.

Exemplary forms of formulation that can be used for topical administration include, but are not limited to, sprays, mists, aerosols, solutions, lotions, gels, serum, creams, ointments, pastes, unguents, emulsions and suspensions. The composition may be in the form of aqueous, aqueous/alcoholic or oily solutions, dispersions of lotion or serum type, aqueous anhydrous or lipophilic gels, emulsions of liquid or semi-liquid consistency of the milk type, obtained by dispersion of a fatty phase in an aqueous phase or conversely an aqueous phase in a fatty phase, or suspensions or emulsions of semi-solid or solid consistency of the cream or gel type, soaps or detergents, or alternatively microemulsions, microcapsules, microparticles, or vesicle dispersions of ionic and/or non-ionic type. Among additional alternative means for topical application of the compositions are spray pumps, aerosol dispersions, impregnated cosmetic facial masks, and impregnated cosmetic facial cloths or sponges.

The composition may be a cleansing, protective, treatment or care cream for the face, for the hands, for the feet, for the major anatomical folds or for the body (for example day creams, night creams, makeup-removing creams, foundation creams, sunscreen creams, fluid foundations, makeup-removing milks, protective or care body milks, anti-sun (sunscreen) milks, skincare lotions, gels or mousses, as cleansing lotions, sunscreen lotions, artificial tanning lotions, bath compositions, deodorizing compositions containing a bactericidal agent, aftershave lotions or gels, hair-removing creams, compositions to combat insect bites and analgesic compositions. The composition can also be formulated as a solid preparation constituting a cleansing bar or a soap. The composition can be formulated as a shampoo or a conditioner, or a toothpaste.

To treat or prevent a cutaneous ulcer(s) and/or non-healing cutaneous wound(s) or a symptom associated therewith, the present composition may be topically applied directly to the affected area in any conventional manner known in the art, e.g., by dropper, applicator stick, or cotton swab, as a mist via an aerosol applicator, via an intradermal or transdermal slow-release patch, or by simply spreading a formulation of the invention onto the affected area with fingers, a sponge, a pad, or wipes. The composition of the present invention can be used topically by rubbing over an area to be treated. A typical method of use is to rub the formulation over the entire area, until the formulation disappears. For liquids formulations, dispensers can include tubes and/or bottles with a sponge or a roll-on applicator such as roller bottles. U.S. Patent Publication No. 20160106690. Additionally, the amount of formulation used can be gradually increased with each successive application.

In certain embodiments, the topically composition are prepared by mixing a pharmaceutically acceptable carrier with the present agent according to known methods in the art, for example, methods provided by standard reference texts such as, Remington: The Science and Practice of Pharmacy 1577-1591, 1672-1673, 866-885 (Alfonso R. Gennaro ed. 19th ed. 1995); Ghosh, T. K.; et al. Transdermal and Topical Drug Delivery Systems (1997), both of which are hereby incorporated herein by reference.

The present composition may contain a gelling agent, a polyol, a protective agent, a cosmetic agent, an adsorbent, a preservative, an antioxidant, a surfactant, a skin-penetration agent, a local anesthetic, an analgesic etc.

Suitable gelling agents known in the art, including those used in the two-phase or single-phase gel systems, can be used in the present invention. Some examples of suitable gelling agents are disclosed in Remington: The Science and Practice of Pharmacy 1517-1518 (Alfonso R. Gennaro ed. 19th ed. 1995), which is hereby incorporated herein by reference. The gelling agents include, but are not limited to, one or more hydrophilic and hydroalcoholic gelling agents used in the cosmetic and pharmaceutical industries. Non-limiting examples of gelling agents include hydroxyethylcellulose, cellulose gum, MVE/MA decadiene crosspolymer, PVM/MA copolymer, glycerine polyacrylate, or a combination thereof. Exemplary hydrophilic gelling agents include carboxyvinyl polymers (carbomer), acrylic copolymers such as acrylate/alkylacrylate copolymers, polyacrylamides, polysaccharides such as hydroxypropylcellulose, natural gums and clays, and, exemplary lipophilic gelling agents include modified clays such as bentones, metal salts of fatty acids such as aluminum stearates, and hydrophobic silica. Exemplary hydrophilic active agents are proteins or protein hydrolysates, amino acids, polyols, urea, allantoin, sugars and sugar derivatives, vitamins and hydroxy acids.

Polyols in gel formulations can serve one or more functions such as solubilizing agents, moisturizers, emollients, skin humectant, skin-penetration agents, etc. Suitable polyols that can be used in embodiments of the present invention include, but are not limited to, glycerine, propylene glycol, dipropylene glycol, hexylene glycol, butylene glycol, and liquid polyethylene glycols, such as polyethylene glycol 200 to 600. Ofher et al., Gels and Jellies, pp. 1327-1344 of Encyclopedia of Pharmaceutical Technology, vol. 3 (ed. by Swarbrick, et al, pub. by Marcel Dekker, 2002); or Pena, “Gel Dosage Forms: Theory, Formulation, and Processing,” pp. 381-388 of Topical Drug Delivery Formulations, (ed. by Osborne et al., pub. by Marcel Dekker, Inc., 1990).

Suitable preservatives include, but are not limited to, quaternary ammonium compounds, such as benzalkonium chloride, benzethonium chloride, cetrimide, dequalinium chloride, and cetylpyridinium chloride; alcoholic agents, for example, chlorobutanol, phenylethyl alcohol, and benzyl alcohol; parabens such as methylparaben, ethylparaben, propylparaben, and butylparaben; antibacterial esters, for example, esters of parahydroxybenzoic acid; and other anti-microbial agents such as chlorhexidine, chlorocresol, benzoic acid, polymyxin, and phenoxyethanol. Preferably, the preservative is selected from the group consisting of sodium benzoate, phenoxyethanol, benzyl alcohol, methylparaben, imidazolidinyl urea and diazolidinyl urea.

Topical administration can continue for about 1 day, about 2 days, about 3 days, about 4 days, about 5 days, about 6 days, about 7 days, about 2 weeks, about 3 weeks, about 4 weeks, about 2 months, about 3 months, about 4 months, about 5 months, about 6 months, about 1 year or longer.

In some embodiments, the present composition may comprise one or more pharmaceutically acceptable antioxidants. Any pharmaceutically acceptable antioxidant known to those of ordinary skill in the art is contemplated for inclusion in the present pharmaceutical compositions. For example, the pharmaceutically acceptable antioxidant may be selected from the group consisting of ascorbic acid, sodium ascorbate, sodium bisulfate, sodium metabisulfate and monothio glycerol.

In some embodiments, the present composition may comprise one or more pharmaceutically acceptable buffering agents. Any pharmaceutically acceptable buffering agent known to those of ordinary skill in the art is contemplated for inclusion in the present pharmaceutical compositions. Examples of such buffering agents include of monobasic sodium phosphate, dibasic sodium phosphate, sodium benzoate, potassium benzoate, sodium citrate, sodium acetate, and sodium tartrate.

The pH of the topical formulations may be within a physiologically acceptable pH, e.g., within the range of about 4 to about 8, of about 6 to about 7.5, or about 4.5 to 6.5.

In some embodiments, the present composition may or may not comprise one or more pharmaceutically acceptable skin penetration enhancers. Examples of such skin penetration enhancers include but not limited to fatty alcohols such as decanol, lauryl alcohol, linolenyl alcohol, n-octanol and oleyl alcohol; fatty acid esters such as ethyl acetate, dodecyl N,N-dimethylamino acetate, glycerol monolaurate, glycerol monooleate, isopropyl myristate, methyl laurate and sorbitan monooleate; fatty acids such as lauric acid and oleic acid; biologics such as lecithin, amines and amides such as N,N-dimethyl-m-toluamide, lauryl-amine and urea; complexing agents such as cyclodextrin, hydroxypropyl methylcellulose and liposomes; surfactants such as Brij 36T, sodium lauryl sulfate and sorbitan monooleate; other compounds such as dimethyl isosorbide, bisabolol, eucalyptol, menthol, terpenes, N-methyl pyrrolidone, azone, DMSO, MSM, decylmethyl sulfoxide, dimethyl formamide, dimethyl acetamide, glycols and propylene glycol.

Exemplary oils that may be used in the present composition, include mineral oils (liquid petroleum jelly), plant oils (liquid fraction of karite butter, sunflower oil), animal oils (perhydrosqualene), synthetic oils (purcellin oil), silicone oils (cyclomethicone) and fluoro oils (perfluoropolyethers). Fatty alcohols and fatty acids (stearic acid) can be added to these oils.

Exemplary emulsifiers that may be used in the present composition, include glyceryl stearate, polysorbate 60 and the mixture PEG-6/PEG-32/glycol stearate.

Representative solvents which can be used include the lower alcohols, such as ethanol and isopropanol.

In certain other embodiments, a surfactant can be used in the present composition, as a wetting agent, emulsifier, solubilizer and/or antimicrobial.

Suitable surfactants include, but are not limited to, sodium stearyl fumarate, diethanolamine cetyl sulfate, polyethylene glycol, isostearate, polyethoxylated castor oil, benzalkonium chloride, nonoxyl 10, octoxynol 9, polyoxyethylene sorbitan fatty acids (polysorbate 20, 40, 60 and 80), sodium lauryl sulfate, sorbitan esters (sorbitan monolaurate, sorbitan monooleate, sorbitan monopalmitate, sorbitan monostearate, sorbitan sesquioleate, sorbitan trioleate, sorbitan tristearate, sorbitan laurate, sorbitan oleate, sorbitan palmitate, sorbitan stearate, sorbitan dioleate, sorbitan sesqui-isostearate, sorbitan sesquistearate, sorbitan tri-isostearate), lecithin pharmaceutical acceptable salts thereof and combinations thereof.

In some embodiments, the topical formulations may contain moisturizing agents. Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol.

### Administration Routes

The present composition may be administered topically, orally, via implant, parenterally, sublingually, rectally, topically, via infiltration, or via inhalation. Injection or implantation includes, but is not limited to subcutaneous (under the skin), intramuscular (muscle), itrathecal, epidural, intraperitoneal, caudal, intradermal or intracutaneous (into the skin), intercostals at a single nerve, intra-articular (joints) or body spaces, intrasynovial (joint fluid), intraspinal (spinal column), intra-arterial (arteries) administrations and administration into other connective tissue compartments. Examples of body spaces include pleura, peritoneium, cranium, mediastinum, pericardium, and bursae or bursal. The present composition may be administered intra-articularly, intra-sternally, intrasynovially, intra-bursally or into body spaces.

### REFERENCES CITED

### U.S. Patent Documents

| | | |
|---|---|---|
| 10,039,770 | August 2018 | Scherer |

### Other References

Alizadeh N, Pepper M S, Modarressi A, Alfo K, Schlaudraff K, Montandon D, et al. Persistent ischemia impairs myofibroblast development in wound granulation tissue: A new model of delayed wound healing. Wound Repair and Regeneration. 2007 15: 809-816.
Bastiaansen-Jenniskens Y M, Wei W, Feijt C, Waarsing J H, Verhaar J A, Zuurmond A M, Hanemaaijer R, Stoop R, van Osch G J. Stimulation of fibrotic processes by the infrapatellar fat pad in cultured synoviocytes from patients with osteoarthritis: a possible role for prostaglandin F2-alpha. Arthritis Rheum. 2013; 65(8):2070-2080.
Borghi V, Bastia E, Guzzetta M, Chiroli V, Toris C B, Batugo M R, Carreiro S T, Chong W K, Gale D C, Kucera D J, Jia L, Prasanna G, Ongini E, Krauss A H, Impagnatiello F. A novel nitric oxide releasing prostaglandin analog, NCX 125, reduces intraocular pressure in rabbit, dog, and primate models of glaucoma. J Ocul Pharmacol Ther. 2010 April; 26(2):125-32. doi: 10.1089/jop.2009.0120.
Boykin J V. Wound nitric oxide bioactivity: a promising diagnostic indicator for diabetic foot ulcer management. J Wound Ostomy Continence Nurs. 2010; 37(1):25-32.
Burrow J W, Koch J A, Chuang H H, Zhong W, Dean D D, Sylvia V L. Nitric oxide donors selectively reduce the expression of matrix metalloproteinases-8 and -9 by human diabetic skin fibroblasts. J Surg Res. 2007; 140(1):90-98.
Ding W Y, Ti Y, Wang Z H, Xie G L, Shang Y Y, Tang M X, Zhang Y, Zhang w, Zhong M. Prostaglandin F2-alpha facilitates collagen synthesis in cardiac fibroblasts via an F-prostanoid receptor/protein kinase C/Rho kinase pathway independent of transforming growth factor □1. Int J Biochem Cell Biol. 2012; 44(6):1031-1039.
Dong J, Ping Y, Wang Y, Zhang Y. The roles of endothelial nitric oxide synthase gene polymorphisms in diabetes mellitus and its associated vascular complications: a systematic review and meta-analysis. Endocrine. 018; 62(2):412-422.
Forstermann U, Munzel T. Endothelial nitric oxide synthase in vascular disease: from marvel to menace. Circulation. 2006; 113(13):1708-1714.
Garcia G A, Ngai P, Mosaed S, Lin K Y. Critical evaluation of latanoprostene bunod in the treatment of glaucoma. Clin Ophthalmol. 2016 Oct. 18; 10:2035-2050.
Harks E G, Scheenen W J, Peters P H, van Zoelen E J, Theuvenet A P. Prostaglandin F2-alpha induces unsynchronized intracellular calcium oscillations in monolayers of gap junctionally coupled NRK fibroblasts. Pfugers Arch. 2003; 447(1):78-86.
Hodges G J, Nawaz S, Tew G A. Evidence that reduced nitric oxide signal contributes to cutaneous microvascular dysfunction in peripheral arterial disease. Clin Hemorheol Microcirc. 2015; 59(1):83-95.
Ignarro L J, Byrns R E, Buga G M, Wood K S. Endothelium-derived relaxing factor from pulmonary artery and vein possesses pharmacologic and chemical properties identical to those of nitric oxide radical. Circ Res. 1987; 61(6):866-879.
Impagnatiello F, Borghi V, Gale D C, Batugo M, Guzzetta M, Brambilla S, Carreiro S T, Chong W K, Prasanna G, Chiroli V, Ongini E, Krauss A H. A dual acting compound with latanoprost amide and nitric oxide releasing properties, shows ocular hypotensive effects in rabbits and dogs. Exp Eye Res. 2011 September; 93(3):243-9. doi: 10.1016/j.exer.2011.02.006. Epub 2011 Feb. 26.
Impagnatiello F, Toris C B, Batugo M, Prasanna G, Borghi V, Bastia E, Ongini E, Krauss A H. Intraocular Pressure-Lowering Activity of NCX 470, a Novel Nitric Oxide-Donating Bimatoprost in Preclinical Models. Invest Ophthalmol Vis Sci. 2015 October; 56(11):6558-64. doi: 10.1167/iovs.15-17190.
Kalinowski L, Matys T, Chabielska E, Buczko W, Malinski T. Angiotensin II AT1 receptor antagonists inhibit platelet adhesion and aggregation by nitric oxide release. Hypertension. 2002; 40(4):521-527.
Kanno Y, Kewashita E, Kokado A, Okada K, Ueshima S, Matsuo O, Matsuno H. Alpha2-antiplasmin regulates the development of dermal fibrosis through adipose triglyceride lipase/calcium-independent phospholipase A(2). Arthritis Rheum. 2013; 65(2):492-502.
Krauss A H, Impagnatiello F, Toris C B, Gale D C, Prasanna G, Borghi V, Chiroli V, Chong W K, Carreiro S T, Ongini E. Ocular hypotensive activity of BOL-303259-X, a nitric oxide donating prostaglandin F2a agonist, in preclinical models. Exp Eye Res. 2011 September; 93(3):250-5. doi: 10.1016/j.exer.2011.03.001.
Leangle U W, Markstein R, Pralet D, Greiner B, Roman D. Effects of latanoprost and GLC756, a noval dopamine D2 agonist and D1 antagomist, on cultured normal human dermal fibroblasts. Eur J Ophthalmol. 2006; 16(1):67-72.
Liu Y, Yanai R, Lu Y, Hirano S, Sagara T, Nishida T. Effects of antiglaucoma drops on collagen gel contraction mediated by human corneal fibroblasts. J Glaucoma, 2006; 15(3):255-259.
Liu Y, Ko J A, Yanai R, Kimura K, Chikama T, Sagara T, Nishida T. Induction by latanoprost of collagen gel contraction mediated by human tenon fibroblasts: role of intracellular signaling molecules. Invest Ophthalmol Vis Sci. 2008; 49(4):1429-1436.
Mikaili P, Maloudizargari M, Aghajanshakeri S. Treatment with topical nitroglycerine may promote the healing process of diabetic foot ulcers. Med Hypothesis. 2014; 83(2):172-174.
Mills J L, Conte M S, Armstrong D G, Pomposelli F B, Schanzer A, Sidawy A N, Andros G. The Society for vascular surgery lower extremity threatened limb classification system: Risk stratification based on wound, ischemia and foot infection. J Vasc Surg. 2013.
Oga T, Matsuoka T, Yao C, Nonomura K, Kitaoka S, Sakata D, Kita Y, Tahizawa K, Taguchi Y, Chin K, Mishima M, Shimizu T, Narumiya S. Prostaglandin F(2-alpha) receptor signaling facilitates bleomycin-induced pulmonary fibrosis independently of transforming growth factor-beta. Nat Med. 2009; 15(12):1426-1430.
Olman M A. Beyond TGF-β: a prostaglandin promotes fibrosis. Nature Med 2009; 15:1360-1361.
Palmer, R M, Ferrige A G, Moncada S. Nitric oxide release accounts for the biological activity of endothelium-derived relaxing factor. Nature. 1987; 327(6122):524-526.
Palmer R M J, Ashton D S, Moncada S. Vascular endothelial cells synthesize nitric oxide from L-arginine. Nature. 1988; 333:664-666.
Rayan G M, Parizi M, Tomasek J J. Pharmacologic regulation of Dupuytren;s fibroblast contraction in vitro. J Hand Surg. 1996; 21(6):1065-1070.
Sadati S M, Radfar M, Hamidi, A K, Abdollahi M, Qorbani M, Esfahani E N, Amoli M M. Association between the polymorphism Glu298Asp in exon 7 of the eNOS gene with foot ulcer and oxidative stress in adult patients with type 2 diabetes. Can J Diabetes. 2018; 42(1):18-22.
Salvemini D, de Nucci G, Gryglewski R J, Vane J R. Human neutrophils and mononuclear cells inhibit platelet aggregation by releasing a nitric-oxide-like factor. Proc Natl Acad Sci USA. 1989; 86(16):6328-6332.
Sinwar P D. The diabetic foot management—recent advances. Int J Surg. 2015 March; 15:27-30. doi: 10.1016/j.ijsu.2015.01.023.
Yang Y, Dengke Y, Wang F, Hou Z, Fang Z. In situ eNOS/NO up-regulation—a simple and effective therapeutic strategy for diabetic skin ulcer. Sci Rep. 2016; 6:30326.
Zhang P, Lu J, Jing Y, Tang S, Zhu D, Bi Y. Global epidemiology of diabetic foot ulceration: a systematic review and meta-analysis. Ann Med. 2017 March; 49(2):106-116. doi: 10.1080/07853890.2016.1231932.

I claim:

1. A method of healing, treating, reducing, preventing, eliminating or curing cutaneous ulcers, non-healing cutaneous wounds or chronic cutaneous and/or subcutaneous wounds via the topical application of an effective amount of a nitric oxide-donating prostaglandin-F2-alpha analog combined with a pharmacologically acceptable carrier, directly to the affected area of a human in need of such treatment.

2. The method of claim 1 in which the nitric oxide-donating prostaglandin-F2-alpha analog is latanoprostene bunod.

3. The method of claim 1 in which the nitric oxide-donating prostaglandin-F2-alpha analog is [(S,E)-1-((1R,2R,3 S,5R)-2-((Z)-7-(ethylamino)-7-oxohept-2-enyl)-3, 5-dihydroxycyclopentyl)-5-phenylpent-1-en-3-yl 6-(nitrooxy)hexanoate].

4. The method of claim 1 in which the prostaglandin-F2-alpha analog or prostamide moiety of the nitric oxide-donating prostaglandin-F2-alpha analog consists of, but is not limited to, prostaglandin-F2-alpha, latanoprost, bimatoprost, travoprost, tafluprost or unoprotone alone or in combination.

5. The method of claim 1 wherein the prostaglandin-F2-alpha analog moiety of the nitric oxide-donating prostaglandin-F2-alpha analog is covalently bound to the nitric oxide-donating moiety.

6. The method of claim 1 wherein the cutaneous ulcer or non-healing wound is due to type I or type II diabetes mellitus, peripheral arterial disease, atherosclerosis, autoimmune vasculopathy, autoimmune vasculitis, sickle cell-related vasculopathy, venous stasis ulcers or any other cause of compromised cutaneous vascular circulation.

7. The method of claim 1 wherein the nitric oxide-donating prostaglandin-F2-alpha analog is formulated as a cream.

8. The method of claim 1 wherein the nitric oxide-donating prostaglandin-F2-alpha analog is formulated as a gel.

9. The method of claim 1 wherein the nitric oxide-donating prostaglandin-F2-alpha analog is formulated as an ointment.

10. The method of claim 1 wherein the nitric oxide-donating prostaglandin-F2-alpha analog is impregnated into a slow-release topical patch, gel or nanoparticle.

11. The method of claim 1 wherein the nitric oxide-donating prostaglandin-F2-alpha analog is formulated as an aerosol spray.

12. The method of claim 1 wherein the nitric oxide-donating prostaglandin-F2-alpha analog is combined with epidermal growth factor (EGF).

13. The method of claim 1 wherein the nitric oxide-donating prostaglandin-F2-alpha analog is combined with a steroid.

14. The method of claim 1 wherein the nitric oxide-donating prostaglandin-F2-alpha analog is combined with a non-steroidal anti-inflammatory agent.

15. The method of claim 1 wherein the nitric oxide-donating prostaglandin-F2-alpha analog is combined with a mast cell stabilizer.

16. The method of claim 1 wherein the nitric oxide-donating prostaglandin-F2-alpha analog is combined with an up-regulator or activator of endogenous endothelial nitric oxide synthase (eNOS).

17. The method of claim 1 wherein the nitric oxide-donating prostaglandin-F2-alpha analog is combined with an antibiotic agent.

18. The method of claim 1 wherein the nitric oxide-donating prostaglandin-F2-alpha analog is combined with an antifungal agent.

19. The method of claim 1 wherein the nitric oxide-donating prostaglandin-F2-alpha analog is combined with micro-needling therapy.

20. The method of claim 1 wherein the nitric oxide-donating prostaglandin-F2-alpha analog is combined with hyperbaric oxygen therapy.
